(19) United States
(12) Patent Application Publication
(10) Pub. No.: US 2004/0192672 A1
(43) Pub. Date: Sep. 30, 2004

(54) Iodobenzopyran-4-one derivatives having fungicidal activity

(76) Inventors: Thomas Wegmann, Saint-Cyr-au Mont-D'or (FR); Nathalie Huser, Lyon (FR); Rainer Karl Preuss, Fontaines-Sur-Saone (FR); Joseph Perez, Lyon (FR)

Correspondence Address: OSTROLENK FABER GERB & SOFFEN, 1180 AVENUE OF THE AMERICAS, NEW YORK, NY, 100368403

(21) Appl. No.: 10/485,337

(22) PCT Filed: Jul. 31, 2002
(86) PCT No.: PCT/EP02/09418

(30) Foreign Application Priority Data
Aug. 3, 2001 (FR) 0110430

(51) Int. Cl. A61K 31/55; A61K 31/443; A61K 31/4025; C 07D 4 5/02

(52) U.S. Cl. 514/217.03; 514/422; 514/456; 514/320; 540/596; 546/196; 548/525; 549/401

(57) ABSTRACT

The present invention relates to iodobenzopyran-4-one derivativespossessing fungicidal properties against phytopathogenic organisms, amethod for preparing some of these derivatives. their use for protecting plants, and fungicidal compositions comprising such derivatives incombination with one or more other lungicidal compounds.

The present invention relates to iodobenzopyran-4-one derivatives possessing fungicidal properties against phytopathogenic organisms, a method for preparing some of these derivatives, their use for protecting plants, and fungicidal compositions comprising such derivatives in combination with one or more other fungicidal compounds.

International Patent Application WO-97/13762 describes in particular derivatives of the halobenzopyranone type. This document discloses a very large number of compounds by means of a very general chemical formula. However, the compounds given as examples are essentially compounds carrying a bromine atom or a carbon chain on the benzene ring. Thus, this document focuses on compounds which are not substituted with an iodine atom as is the case of the present invention. Indeed, only two iodinated compounds are disclosed by this document without however indicating that such compounds can possess properties which are superior to the other derivatives, in particular the brominated derivatives.

It has now been discovered that certain particular iodobenzopyran-4-one derivatives possess excellent fungicidal properties against phytopathogenic organisms. These properties may be further improved when these compounds are used as a mixture with one or more other fungicidal compounds.

A first aspect of the present invention relates to the compounds of formula (I)

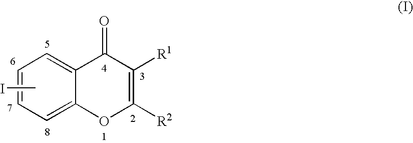

in which:

the iodine atom is placed in the 5, 6, 7 or 8 position;

R′ is chosen from a halogen atom, a substituted or unsubstituted C₁-C₆ alkyl radical, a substituted or unsubstituted C₁-C₆ alkenyl radical and a substituted or unsubstituted C₁-C₆ alkynyl radical;

R² is chosen from a substituted or unsubsbtuted C₁-C₆ alkyl radical, a substituted or unsubstituted C₁-C₆alkenyl radical and a substituted or unsubstituted C₁-C₆ alkynyl radical, a 3- to 7-membered carbo- or heterocycle which is substituted or unsubstituted and which may be saturated, unsaturated or aromatic, a halogen atom, the cyano radical, the radical —W—R³;

W is chosen from oxygen, sulphur or the radical —NR⁴;

R³ and R⁴, which are identical or different, are chosen, independently of each other, from the hydrogen atom, a substituted or unsubstituted C₁-C₆ alkyl radical, a substituted or unsubstituted C₁-C₆ alkenyl radical and a substituted or unsubstituted C₁-C₆ alkynyl radical, an alkoxy radical, an amino radical, a 3- to 7-membered carbo- or heterocycle which is substituted or unsubstituted and which may be saturated, unsaturated or aromatic, R³ and R⁴ may form together a 5- to 7-membered heterocycle which may be saturated, unsaturated or aromatic, and which may also comprise another heteroatom; and their possible geometric and/or optical isomers, in pure form or in the form of mixtures, in any proportions, including the racemic mixture(s), their possible N-oxides, addition salts with an acid, and their possible metal or metalloid complexes; R³ being different from the methyl radical or the butyl radical when the iodine is in the 6-position, when R¹ is the n-propyl radical and when W represents oxygen.

When the radicals of the compounds of formula (I) are substituted, they are substituted in a preferred manner with one or more groups which may be chosen independently of each other from alkyl, alkenyl and alkynyl radicals, a halogen atom, the cyano, trialkylsilyl, alkoxy, alkylthio, hydroxyl, nitro, amino, acyl, acyloxy, phenyl, heterocyclyl, phenylthio, phenoxy, heterocyclyloxy or heterocyclylthio radical and oxidized derivatives and may be optionally substituted with chemical entities containing a thio group.

The term heterocyclyl comprises heteroaryl groups and non-aromatic heterocyclyl groups which may be saturated or unsaturated.

The heteroaryl groups are generally 5- or 6-membered rings containing up to 4 heteroatoms chosen from nitrogen, oxygen and sulphur, optionally fused with a benzene ring. As examples of heteroaryl groups, there especially may be mentioned groups derived from thiophene, furan, pyrrole, thiazole, oxazole, imidazole, isothiazole, isoxazole, pyrazole, 1,3,4-oxadiazole, 1,3,4-thiadiazole, 1,2,4-oxadiazole, 1,2,4-thiadiazole, 1,2,4-triazole, 1,2,3-triazole, tetrazole, benzo[b]thiophene, benzo[b]furan, indole, benzo[c]thiophene, benzo[c]furan, isoindole, benzoxazole, benzothiazole, benzimidazole, benzisoxazole, benzisothiazole, indazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-tetrazine, quinoline, isoquinoline, quinoxaline, quinazoline, cinnoline, 1,8-naphthyridine, 1,5-naphthyridine, 1,6-naphthyridine, 1,7-naphthyrdine, phthalazine, pyridopyrimidine, purine or pteridine.

The non-aromatic heterocyclyl groups are generally 3-, 5-, 6- or 7-membered rings containing up to 3 heteroatoms chosen from nitrogen, oxygen and sulphur, for example oxiranyl, thiiranyl, thiazolinyl, dioxolanyl, 1,3-benzoxazinyl, 1,3-benzothiazinyl, morpholino, pyrazolinyl, sulpholanyl, dihydroquinazolinyl, piperidinyl, phthalimido, tetrahydrofuranyl, tetrahydropyranyl, pyrrolidinyl, indolinyl, 2-oxopyrrolidino, 2-oxobenzoxazolin-3-yl or tetrahydroazepinyl.

The substituents, when they are present, on the phenyl or heterocyclyl groups may, for example, be halogen atoms, CN, NO₂, SF₅, B(OH)₂, trialkylsilyl, acyl, O-acyl or a radical E, OE or S(O)ₙ E as defined above for R² or alternatively is an optionally substituted amino radical; or alternatively two adjacent groups on the ring, together with the atoms to which they are attached, form a carbocyclic or heterocyclic ring which may be optionally substituted in a similar manner.

The term acyl comprises the acid residues containing sulphur or phosphorus and the carboxylic acid residues. Examples of acyl groups are thus —COR⁵, —COOR⁵, —ClNR⁵R⁶, —CON(R⁵)OR⁶, —COONR⁵R⁶, —CON(R⁵)NR⁶R⁷, —COSR⁵, —CSSR⁵, —S(O)_{q}R⁵, —S(O)₂OR⁵, —S(O)_{q}NR⁵R⁶, —P(═L)(OR⁵)(OR⁶) or —COOR⁵, in which R⁵, R⁶ and R⁷, which may be identical or different, represent the hydrogen atom, an optionally substituted alkyl radical, an optionally substituted cycloalkyl radical, an optionally substituted cycloalkenyl radical, an optionally substituted alkenyl radical, an optionally substituted alkynyl radical, an optionally substituted phenyl group or an optionally substituted heterocyclyl group, or alternatively R⁵ and R⁶, or R⁶ and R⁷, together with the atom(s) to which they are attached, may form a ring, q represents 1 or 2 and L represents O or S.

The amino radicals may be substituted, for example, with one or two optionally substituted alkyl or optionally substituted acyl radicals, or alternatively two substituents may form a ring, preferably a 5- to 7-membered ring, which may be substituted and which may contain other heteroatoms, and for example morpholine.

Among the compounds of formula (I) there are preferred those possessing at least one of the following characteristics:

the iodine atom is in the 6-position;

R¹ represents a C₂-C₄ alkyl radical;

R² represents the radical —W—R³ in which W represents oxygen and R³ is as defined above.

The most preferred compounds of formula (I) simultaneously possess these three characteristics, in particular the compounds of formula (I) in which R³ represents a C₁-C₆ alkyl radical, a C₁-C₆ alkenyl radical or a C₁-C₆ alkynyl radical or alternatively the compounds of formula (I) in which R¹ is a n-propyl radical.

Among the compounds of formula (I), there may be mentioned, by way of examples, the following compounds of formulae (Ia) and (Ib) which do not limit the scope of the present invention:

(Ia)

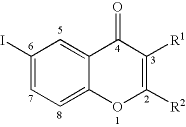

| Example | R1 | R2 |
|---|---|---|
| 1 | 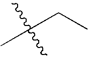 | 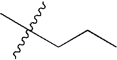 |
| 2 | 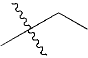 | 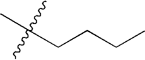 |
| 3 | 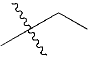 | 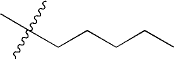 |
| 4 | 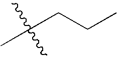 | 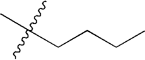 |
| 5 | 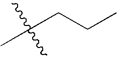 | 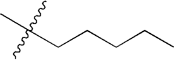 |
| 6 | 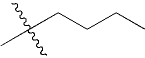 | 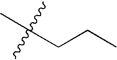 |
| 7 | 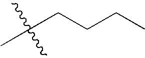 | 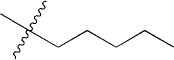 |
| 8 | 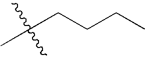 | 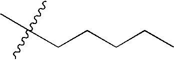 |
| 9 | 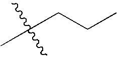 | 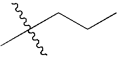 |

(Ib)

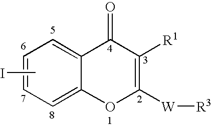

| Ex- | | | | |
|---|---|---|---|---|
| am- | | | | |
| ple | I | W | R1 | R3 |
| 10 | 8-I | O | 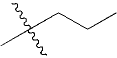 | 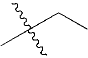 |
| 11 | 6-I | O | 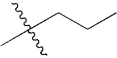 | 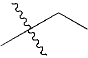 |
| 12 | 6-I | O | 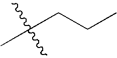 | 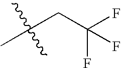 |
| 13 | 6-I | O | 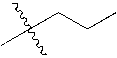 | 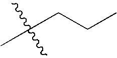 |
| 14 | 6-I | O | 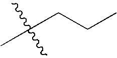 | 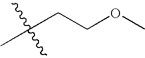 |
| 15 | 6-I | O | 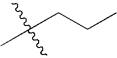 | 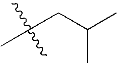 |
| 16 | 6-I | O | 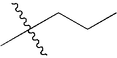 | 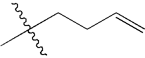 |
| 17 | 6-I | O | 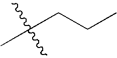 | 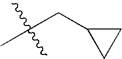 |
| 18 | 6-I | O | 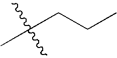 | 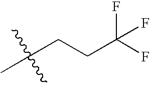 |
| 19 | 6-I | O | 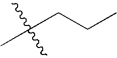 | 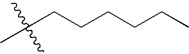 |
| 20 | 6-I | O | 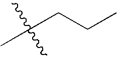 | 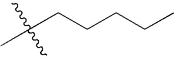 |
| 21 | 6-I | O | 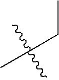 | 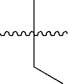 |
| 22 | 6-I | O | 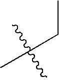 | 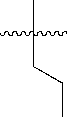 |
| 23 | 6-I | O | 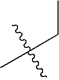 | 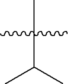 |
| 24 | 6-I | O | 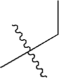 | 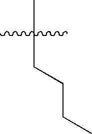 |
| 25 | 6-I | O | 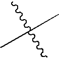 | 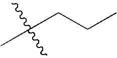 |
| 26 | 6-I | O | 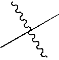 | 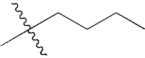 |
| 27 | 6-I | O | 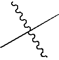 | 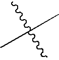 |
| 28 | 6-I | O | 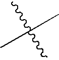 | 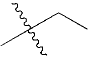 |
| 29 | 6-I | O | 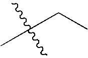 | 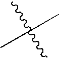 |
| 30 | 6-I | O | 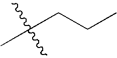 | 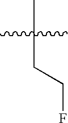 |
| 31 | 6-I | O | 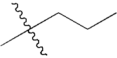 | 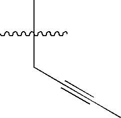 |
| 32 | 6-I | O | 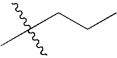 | 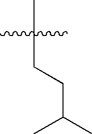 |
| 33 | 6-I | O | 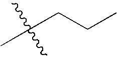 | 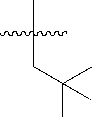 |
| 34 | 6-I | O | 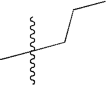 | 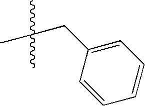 |
| 35 | 6-I | O | 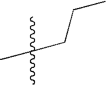 | 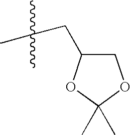 |
| 36 | 6-I | O | 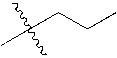 | 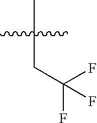 |
| 37 | 6-I | O | 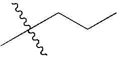 | 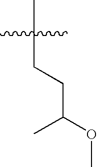 |
| 38 | 6-I | O | 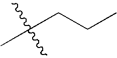 | 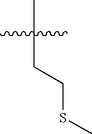 |
| 39 | 6-I | O | 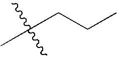 | 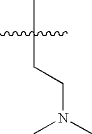 |
| 40 | 6-I | O | 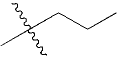 | 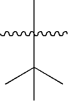 |
| 41 | 6-I | O | 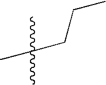 | 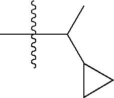 |
| 42 | 6-I | O | 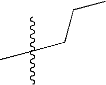 | 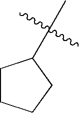 |
| 43 | 6-I | O | 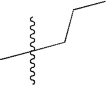 | 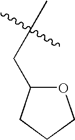 |
| 44 | 6-I | O | 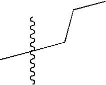 | 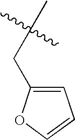 |
| 45 | 6-I | O | 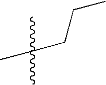 | 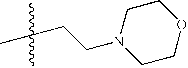 |
| 46 | 6-I | O | 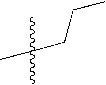 | 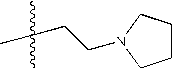 |
| 47 | 6-I | O | 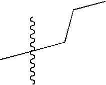 | 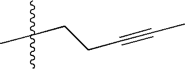 |
| 48 | 6-I | O | 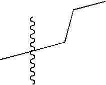 | 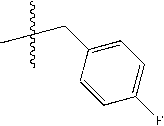 |
| 49 | 6-I | O | 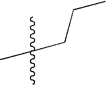 | 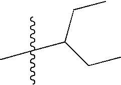 |
| 50 | 6-I | O | 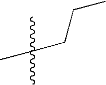 | 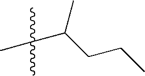 |
| 51 | 6-I | O | 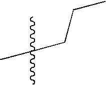 | 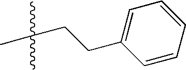 |
| 52 | 6-I | O | 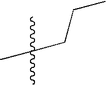 | 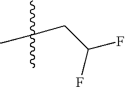 |
| 53 | 6-I | O | 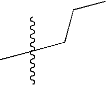 | 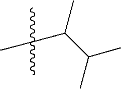 |
| 54 | 6-I | O | 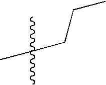 | 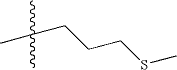 |
| 55 | 6-I | O | 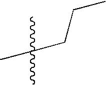 | 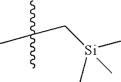 |
| 56 | 6-I | O | 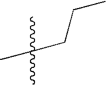 | 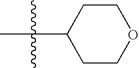 |
| 57 | 6-I | O | 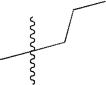 | 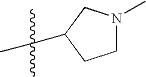 |
| 58 | 6-I | O | 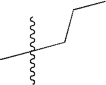 | 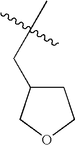 |
| 59 | 6-I | O | 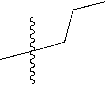 | 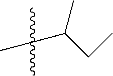 |
| 60 | 6-I | O | 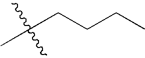 | 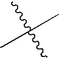 |
| 61 | 6-I | O | 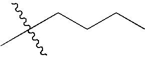 | 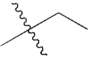 |
| 62 | 6-I | O | 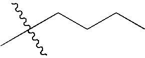 | 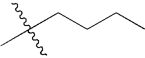 |
| 63 | 6-I | O | 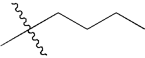 | 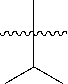 |
| 64 | 6-I | O | 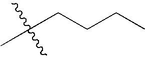 | 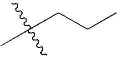 |
| 65 | 6-I | O | 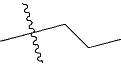 | 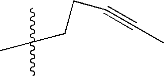 |
| 66 | 6-I | O | 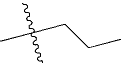 | 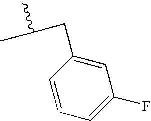 |
| 67 | 6-I | O | 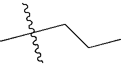 | 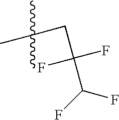 |
| 68 | 6-I | O | 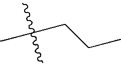 | 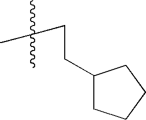 |
| 69 | 6-I | O | 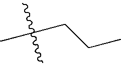 | 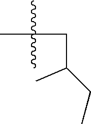 |
| 70 | 6-I | O | 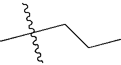 | 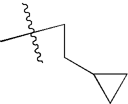 |
| 71 | 6-I | O | 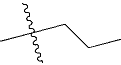 | 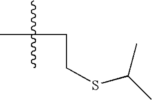 |
| 72 | 6-I | O | 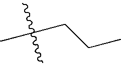 | 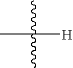 |
| 73 | 6-I | O | 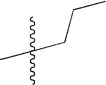 | 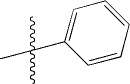 |
| 74 | 6-I | O | 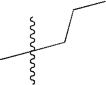 | 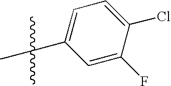 |
| 75 | 6-I | O | 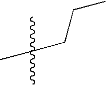 | 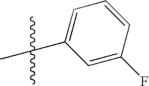 |
| 76 | 6-I | O | 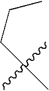 | 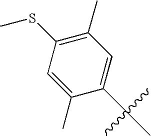 |
| 77 | 6-I | O | 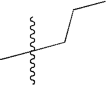 | 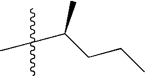 |
| 78 | 6-I | O | 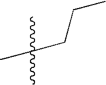 | 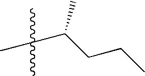 |
| 79 | 6-I | O | 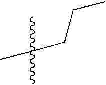 | 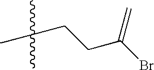 |
| 80 | 6-I | O | 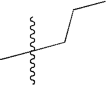 | 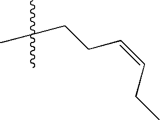 |
| 81 | 6-I | O | 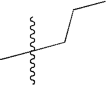 | 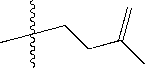 |
| 82 | 6-I | O | 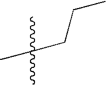 | 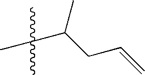 |
| 83 | 6-I | O | 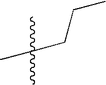 | 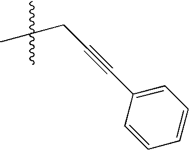 |
| 84 | 6-I | O | 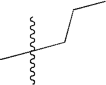 | 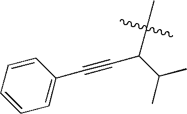 |
| 85 | 6-I | O | 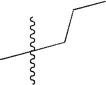 | 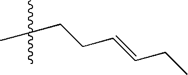 |
| 86 | 6-I | O | 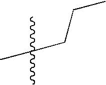 | 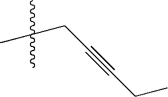 |
| 87 | 6-I | O | 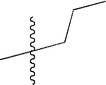 | 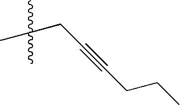 |
| 88 | 6-I | O | 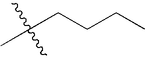 | 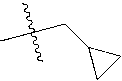 |
| 89 | 6-I | O | 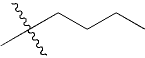 | 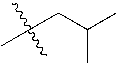 |
| 90 | 6-I | O | 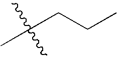 | 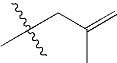 |
| 91 | 6-I | O | 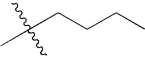 | 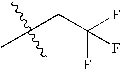 |
| 92 | 6-I | O | 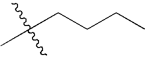 | 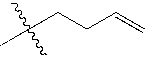 |
| 93 | 6-I | O | 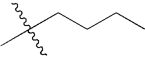 | 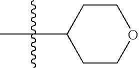 |
| 94 | 6-I | O | 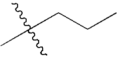 | 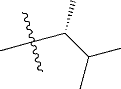 |
| 95 | 6-I | O | 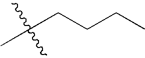 | 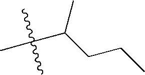 |
| 96 | 6-I | O | 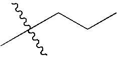 | 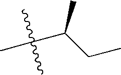 |
| 97 | 6-I | O | 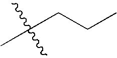 | 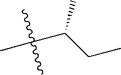 |
| 98 | 6-I | NR4 | 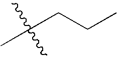 | R3═R4═H |

The compounds of the present invention may be prepared by numerous methods which are known procedures, in particular those disclosed in Patent Application EP-861 242.

In a particularly advantageous manner, the compounds of formula (I) may also be prepared according to the following method:

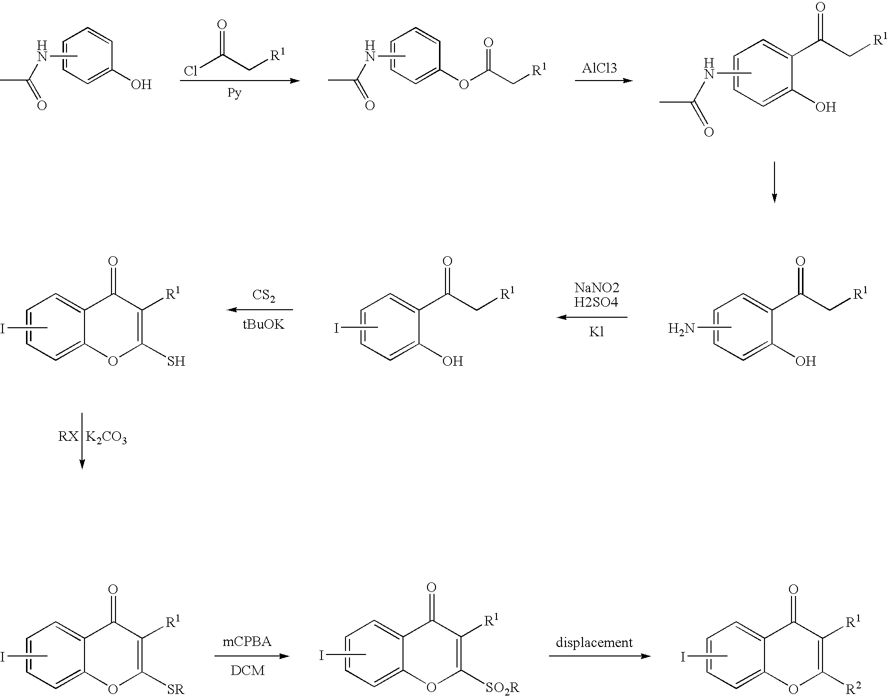

This method constitutes another aspect of this invention.

Other methods may also be used to prepare the compounds of formula (I), such as methods similar to those described in Chemistry and Industry, (1980), 116; J. Chem. Soc. Chem. Com., 1, (1981), 282 and J. Org. Chem., (1992), 57, 6502.

The reagents and some of the intermediate compounds useful for the preparation of the compounds of formula (I) may be prepared by methods known to persons skilled in the art.

Another aspect of the present invention relates to a fungicidal composition comprising

a) a compound of formula (I):

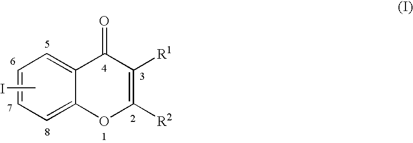

in which:

the iodine atom is placed in the 5, 6, 7 or 8 position;

R¹ is chosen from a halogen atom, a substituted or unsubsbtuted C₁-C₆ alkyl radical, a substituted or unsubstituted C₁-C₆ alkenyl radical and a substituted or unsubstituted C₁-C₆ alkynyl radical;

R² is chosen from a substituted or unsubstituted C₁-C₆ alkyl radical, a substituted or unsubstituted C₁-C₆ alkenyl radical and a substituted or lo unsubstituted C₁-C₆ alkynyl radical, a 3- to 7-membered carbo- or heterocycle which is substituted or unsubstituted and which may be saturated, unsaturated or aromatic, a halogen atom, the cyano radical, the radical —W—R³;

W is chosen from oxygen, sulphur or the radical —NR⁴;

R³ and R⁴, which are identical or different, are chosen, independently of each other, from the hydrogen atom, a substituted or unsubstituted C₁-C₆ alkyl radical, a substituted or unsubstituted C₁-C₆ alkenyl radical and a substituted or unsubstituted C₁-C₆ alkynyl radical, an alkoxy radical, an amino radical, a 3- to 7-membered carbo- or heterocycle which is substituted or unsubstituted and which may be saturated, unsaturated or aromatic, R³ and R⁴ may form together a 5- to 7-membered heterocycle which may be saturated, unsaturated or aromatic, and which may also comprise another heteroatom; and their possible geometric and/or optical isomers, in pure form or in the form of mixtures, in any proportions, including the racemic mixture(s), their possible N-oxides, addition salts with an acid, and their possible metal or metalloid complexes;

b) at least one other fungicidal compound.

The preferred compounds of formula (I) for the composition according to the invention are those possessing at least one of the following characteristics:

the iodine atom is in the 6-position;

R¹ represents a C₂-C₄ alkyl radical;

R² represents the radical —W—R³ in which W represents oxygen and R³ is as defined above.

The most preferred compounds of formula (I) for the composition according to the invention simultaneously possess these three characteristics, in particular the compounds of formula (I) in which R³ represents a C₁-C₆ alkyl radical, a C₁-C₆ alkenyl radical or a C₁-C₆ alkynyl radical or alternatively the compounds of formula (I) in which R¹ is a n-propyl radical.

There may be mentioned in particular the compounds whose chemical name is the following:

2-butoxy-6-iodo-3-propyl-benzopyran-4-one,

2-ethoxy-6-iodo-3-propyl-benzopyran-4-one,

6-iodo-2-propoxy-3-propyl-benzopyran-4-one,

2-but-2-ynyloxy-6-iodo-3-propyl-benzopyran-4-one,

6-iodo-2-(1-methyl-butoxy)-3-propyl-benzopyran4-one,

2-but-3-enyloxy-6-iodo-3-propyl-benzopyran-4-one,

3-butyl-6-iodo-2-isopropoxy-benzopyran-4-one,

6-iodo-3-propyl-2-(tetrahydro-pyran-4-yloxy)-benzopyran-4-one,

6-iodo-3-propyl-2-(2,2,2-trifluro-ethoxy)-benzopyran-4-one.

In the composition according to the invention, there is preferred the use of fungicidal compounds chosen from

b1) compounds capable of inhibiting the transport of electrons in the mitochondrial ubiquinol:ferricytochrome-c oxidoreductase respiratory chain of phytopathogenic fungal organisms, in particular strobilurin derivatives such as azoxystrobin, dimoxystrobin, fluoxastrobin, kresoxim-methyl, pycoxystrobin, pyraclostrobin, trifloxystrobin, or fenamidone or famoxadone; or

b2) the compounds capable of inhibiting the biosynthesis of ergosterol, in particular compounds of the triazole type such as bromuconazole, epoxyconazole, fluquinconazole, prochloraz, prothioconazole, tebuconazole, triadimefon, triadimenol, triticonazole.

As other preferred compounds which may be used in the composition according to the invention, there may be mentioned cyprodinil, dinocap, fenpropidin, fenpropimorph, fosetyl, iprovalicarb, quinoxyfen, spiroxamine.

As particular combinations of compounds of formula (I) with one or more other fungicidal compounds, the following combinations are preferred:

| Compounds of formula (I): | Compound(s) b: |
|---|---|
| 6-iodo-2-(1-methyl-butoxy)-3-propyl-benzopyran-4-one | + trifloxystrobin |
| 6-iodo-2-(1-methyl-butoxy)-3-propyl-benzopyran-4-one | + pyraclostrobin |
| 6-iodo-2-(1-methyl-butoxy)-3-propyl-benzopyran-4-one | + picoxystrobin |
| 6-iodo-2-(1-methyl-butoxy)-3-propyl-benzopyran-4-one | + kresoxym-methyl |
| 6-iodo-2-(1-methyl-butoxy)-3-propyl-benzopyran-4-one | + fluquinconazole |
| 6-iodo-2-(1-methyl-butoxy)-3-propyl-benzopyran-4-one | + tebuconazole |
| 6-iodo-2-(1-methyl-butoxy)-3-propyl-benzopyran-4-one | + prochloraz |
| 6-iodo-2-(1-methyl-butoxy)-3-propyl-benzopyran-4-one | + prothioconazole |
| 6-iodo-2-(1-methyl-butoxy)-3-propyl-benzopyran-4-one | + triadimefon |
| 6-iodo-2-(1-methyl-butoxy)-3-propyl-benzopyran-4-one | + triadimenol |
| 6-iodo-2-(1-methyl-butoxy)-3-propyl-benzopyran-4-one | + triticonazole |
| 6-iodo-2-(1-methyl-butoxy)-3-propyl-benzopyran-4-one | + epoxiconazole |
| 6-iodo-2-(1-methyl-butoxy)-3-propyl-benzopyran-4-one | + dinocap |
| 6-iodo-2-(1-methyl-butoxy)-3-propyl-benzopyran-4-one | + spiroxamine |
| 6-iodo-2-(1-methyl-butoxy)-3-propyl-benzopyran-4-one | + fenpropidin |
| 6-iodo-2-(1-methyl-butoxy)-3-propyl-benzopyran-4-one | + fenpropimorph |
| 6-iodo-2-(1-methyl-butoxy)-3-propyl-benzopyran-4-one | + quinoxyfen |
| 6-iodo-2-(1-methyl-butoxy)-3-propyl-benzopyran-4-one | + cyprodinil |
| 6-iodo-2-(1-methyl-butoxy)-3-propyl-benzopyran-4-one | + fosetyl-Al |
| 6-iodo-2-(1-methyl-butoxy)-3-propyl-benzopyran-4-one | + fenamidone |
| 6-iodo-2-(1-methyl-butoxy)-3-propyl-benzopyran-4-one | + iprovalicarb |
| 6-iodo-2-(1-methyl-butoxy)-3-propyl-benzopyran-4-one | + fluoxastrobin |
| 2-butoxy-6-iodo-3-propyl-benzopyran-4-one | + trifloxystrobin |
| 2-butoxy-6-iodo-3-propyl-benzopyran-4-one | + pyraclostrobin |
| 2-butoxy-6-iodo-3-propyl-benzopyran-4-one | + picoxystrobin |
| 2-butoxy-6-iodo-3-propyl-benzopyran-4-one | + kresoxym-methyl |
| 2-butoxy-6-iodo-3-propyl-benzopyran-4-one | + fluquinconazole |
| 2-butoxy-6-iodo-3-propyl-benzopyran-4-one | + tebuconazole |
| 2-butoxy-6-iodo-3-propyl-benzopyran-4-one | + prochloraz |
| 2-butoxy-6-iodo-3-propyl-benzopyran-4-one | + prothioconazole |
| 2-butoxy-6-iodo-3-propyl-benzopyran-4-one | + triadimefon |
| 2-butoxy-6-iodo-3-propyl-benzopyran-4-one | + triadimenol |
| 2-butoxy-6-iodo-3-propyl-benzopyran-4-one | + triticonazole |
| 2-butoxy-6-iodo-3-propyl-benzopyran-4-one | + epoxiconazole |
| 2-butoxy-6-iodo-3-propyl-benzopyran-4-one | + dinocap |
| 2-butoxy-6-iodo-3-propyl-benzopyran-4-one | + spiroxamine |
| 2-butoxy-6-iodo-3-propyl-benzopyran-4-one | + fenpropidine |
| 2-butoxy-6-iodo-3-propyl-benzopyran-4-one | + fenpropimorphe |
| 2-butoxy-6-iodo-3-propyl-benzopyran-4-one | + quinoxyfen |
| 2-butoxy-6-iodo-3-propyl-benzopyran-4-one | + cyprodinil |
| 2-butoxy-6-iodo-3-propyl-benzopyran-4-one | + fosetyl-al |
| 2-butoxy-6-iodo-3-propyl-benzopyran-4-one | + fenamidone |
| 2-butoxy-6-iodo-3-propyl-benzopyran-4-one | + iprovalicarb |
| 2-butoxy-6-iodo-3-propyl-benzopyran-4-one | + fluoxastrobin |
| 2-butoxy-6-iodo-3-propyl-benzopyran-4-one | + trifloxystrobine |
| | + fluquinconazole |
| 2-butoxy-6-iodo-3-propyl-benzopyran-4-one | + trifloxystrobine |
| | + tebuconazole |
| 2-butoxy-6-iodo-3-propyl-benzopyran-4-one | + trifloxystrobine |
| | + prochloraz |
| 2-butoxy-6-iodo-3-propyl-benzopyran-4-one | + trifloxystrobine |
| | + prothioconazole |
| 2-butoxy-6-iodo-3-propyl-benzopyran-4-one | + trifloxystrobine |
| | + spiroxamine |
| 2-butoxy-6-iodo-3-propyl-benzopyran-4-one | + fluoxastrobin |
| | + fluquinconazole |
| 2-butoxy-6-iodo-3-propyl-benzopyran-4-one | + fluoxastrobin |
| | + tebuconazole |
| 2-butoxy-6-iodo-3-propyl-benzopyran-4-one | + fluoxastrobin |
| | + prochloraz |
| 2-butoxy-6-iodo-3-propyl-benzopyran-4-one | + fluoxastrobin |
| | + prothioconazole |
| 2-butoxy-6-iodo-3-propyl-benzopyran-4-one | + fluoxastrobin |
| | + spiroxamine |
| 2-butoxy-6-iodo-3-propyl-benzopyran-4-one | + fosetyl-al |
| | + fenamidone |
| 6-iodo-2-(1-methyl-butoxy)-3-propyl-benzopyran-4-one | + trifloxystrobine |
| | + fluquinconazole |
| 6-iodo-2-(1-methyl-butoxy)-3-propyl-benzopyran-4-one | + trifloxystrobine |
| | + tebuconazole |
| 6-iodo-2-(1-methyl-butoxy)-3-propyl-benzopyran-4-one | + trifloxystrobine |
| | + prochloraz |
| 6-iodo-2-(1-methyl-butoxy)-3-propyl-benzopyran-4-one | + trifloxystrobine |
| | + prothioconazole |
| 6-iodo-2-(1-methyl-butoxy)-3-propyl-benzopyran-4-one | + trifloxystrobine |
| | + spiroxamine |
| 6-iodo-2-(1-methyl-butoxy)-3-propyl-benzopyran-4-one | + fluoxastrobin |
| | + fluquinconazole |
| 6-iodo-2-(1-methyl-butoxy)-3-propyl-benzopyran-4-one | + fluoxastrobin |
| | + tebuconazole |
| 6-iodo-2-(1-methyl-butoxy)-3-propyl-benzopyran-4-one | + fluoxastrobin |
| | + prochloraz |
| 6-iodo-2-(1-methyl-butoxy)-3-propyl-benzopyran-4-one | + fluoxastrobin |
| | + prothioconazole |
| 6-iodo-2-(1-methyl-butoxy)-3-propyl-benzopyran-4-one | + fluoxastrobin |
| | + spiroxamine |
| 6-iodo-2-(1-methyl-butoxy)-3-propyl-benzopyran-4-one | + fosetyl-Al |
| | + fenamidone |

It has been possible to demonstrate a synergistic effect linked to the use of the composition according to the invention.

For their use, the compounds of formula (I) and the compositions comprising a compound of formula (I) combined with one or more other fungicidal compounds are usually mixed with a carrier and a surfactant which are acceptable for use in agriculture.

The carrier or diluent in the composition according to the present invention may be a solid or a liquid, optionally in combination with a surfactant, for example a dispersing agent, an emulsifying agent or a wetting agent. Suitable surfactants comprise anionic compounds such as a carboxylate, for example a metal carboxylate having a long chain fatty acid; a N-acylsarcosinate; mono- or diesters of phosphoric acid with fatty alcohol ethoxylates or alternatively salts of the said esters; fatty alcohol sulphates such as sodium dodecyl sulphate, sodium octadecyl sulphate or sodium cetyl sulphate; ethoxylated fatty alcohol sulphates; ethoxylated alkylphenol sulphates; lignosulphonates; petroleum sulphonates; alkylaryl sulphonates such as alkylbenzene sulphonates or low alkylnaphthalene sulphonates, for example butyinaphthalene sulphonates; salts of sulphonated naphthalene-formaldehyde condensates; salts of sulphonated phenol-formaldehyde condensates; or even more complex sulphonates such as amidesulphonates, for example the sulphonated product of condensation of oleic acid with N-methyltaurine or dialkyl sulphosuccinates, for example sodium sulphonate of dioctyl succinate. Among the nonionic agents, there may be mentioned the products of condensation of esters of fatty acids, of fatty alcohols, of amides of fatty acids or of phenols substituted by fatty alkyls or alkenyls with ethylene oxide, fatty esters of ethers of polyhydric alcohols, for example sorbitan fatty acid esters, products of condensation of the said esters with ethylene oxide, for example fatty acid esters of polyoxyethylene sorbitan, block copolymers of ethylene and propylene oxide, acetylenic glycols such as 2,4,7,9-tetramethyl-5-decyne-4,7-diol, or ethoxylated acetylenic glycols.

Among the cationic surfactants, there may be mentioned, for example, an aliphatic mono-, di- or polyamine in acetate, naphthenate or oleate form; an oxygen-containing amine such as an amine oxide or a polyoxyethylene alkylamine; an amine containing an amide bond prepared by condensation of a carboxylic acid with a diamine or a polyamine; or a quaternary ammonium salt.

The compositions according to the present invention may take any forms known in the art of formulating agrochemical compounds, for example, a solution, dispersion, aqueous emulsion, pulverulent powder, formulation for treating seeds, formulation for fumigation or smoking, dispersible powder, emulsifiable concentrate or granules. Furthermore, they may be in a form suitable for direct use or in a concentrated form or in the form of a primary composition requiring dilution with a suitable quantity of water or another diluent before application.

The concentration of active ingredient(s) in the composition of the present invention, as applied to plants, is preferably in the range between 0.0001 to 1.0% by weight, particularly 0.0001 and 0.01% by weight. In a primary composition, the quantity of active ingredient may vary considerably and may be, for example, between 5 and 95% by weight of composition.

The compounds of formula (I) of the present invention, used alone or in the form of mixtures, possess activity as a fungicide, particularly against fungicidal diseases of plants, for example powdery mildews and downy mildews and particularly cereal powdery mildew (Blumeria graminis), vine powdery mildew (Uncinula necator), apple powdery mildew (Podosphaera Ieucotricha), cucurbit powdery mildews (for example Erysiphe cichoracearum, Sphaerotheca fuliginea, Erysiphe polygoni), powdery mildew of Solanaceae (for example Leveillula taurica), powdery mildew of fruit and ornamental plants (for example Sphaerotheca pannosa), vine downy mildew (Plasmopara viticola), rice blight (Pyricularia oryzae), cereal eyespot (Pseudocercosporella herpotrichoides), rice sheath blight (Pellicularia sasakii), grey mould (Botrytis cinerea), damping off (Rhizoctonia solani), wheat brown rust (Puccinia recondita), tomato or potato blight (Phytophthora infestans), apple scab (Venturia inaequalis), glume blotch (Leptosphaeria nodorum).

The compounds of formula (I) may also prove to be active on other phytopathogenic fungi, including other types of powdery mildew, rusts, as well as general pathogens originating from Deuteromycetes, Ascomycetes, Phycomycetes and Basidiomycetes.

The compounds of formula (I) according to the present invention have proved particularly active against powdery mildews of cereals, vine, plants and fruit trees, vegetable crops and ornamental plants.

Another aspect of the invention therefore also relates to a method for controlling phytopathogenic fungi of crops at a site which is infested or which may be infested by them, which comprises the application to the said site of at least one compound of formula (I), alone or in combination with one or more other fungicidal compounds as described above, notably within a composition according to the present invention.

In the method according to the present invention, the compound of formula (I) is generally applied to the seeds, the plants or to the place where they grow or will grow. Thus, this compound may be applied directly to the soil before, at the time of, or after sowing such that the presence of the active ingredient in the soil can control the growth of the phytopathogenic fungi which can attack seeds. When the soil is treated directly, the active substance may be applied in any manner so that it is intimately mixed with the soil, for example by spraying, by land spreading of a solid form such as granules, or by applying the active ingredient at the time of sowing by incorporating it into the seeds in the seeder. A suitable dose for application is in the range from 5 to 1 000 g per hectare, preferably from 10 to 500 g per hectare.

An alternative consists in applying the active ingredient directly to the plant, for example by spraying or dusting, either when the phytopathogenic fungus has started to appear on the plant, or before the appearance of the said fungus as a preventive measure, or preventively and curatively. In each of these cases, the preferred method of application is foliar spraying. It is generally important to obtain good control of phytopathogenic fungi during the first stages of plant growth, these stages being the precise moments when the plants may be the most seriously damaged. It is sometimes advantageous to treat the roots of the plant before or during planting, for example by dipping the roots in a composition comprising a compound of formula (I).

The invention is illustrated without limitation by the following examples.

### EXAMPLE OF TEST N^{o}1

The compounds were tested for their activity against wheat powdery mildew (Blumeda graminis).

Winter wheat (Appolo variety) was planted in France (department of Marne) in 1 m² plots, at the rate of 200 kg/ha (soil: coloured rendzina) at a depth of 3 cm. The compounds of the invention were applied to the wheat plants in two portions: Application A at the 1 cm ear stage and then Application B at the second visible node stage. Each of these applications was carried out by spraying an aqueous solution of the compounds at a dose of 125 g of active ingredient/ha.

During Application A, the intensity of attack by the fungus Blumeria graminis is estimated at 3% of the total foliar volume.

The tests of intensity of attack are carried out 52 days after Application B by evaluating the percentage of affected surface area of the second leaves (counted from the ear).

The results presented in Table 1 were obtained with the following compounds according to the invention:

Compound 1: 2-ethoxy-6-iodo-3-propylbenzopyran-4-one

Compound 2: 6-iodo-2-propoxy-3-propylbenzopyran-4-one

Compound 3: 2-butoxy-6-iodo-3-propylbenzopyran-4-one

Control: untreated plants and Compound B: quinoxyfen (commercial reference Fortress®).

TABLE 1
(Test of intensity of attack)

| | % affected surface |
|---|---|
| Compound | area per (second) leaf |
| 1 | 0.56 |
| 2 | 2.53 |
| 3 | 2.51 |
| Control | 30.47 |
| A | 6.47 |
| B | 4.91 |

Under the same conditions, a test of frequency of attack was performed by evaluating the percentage of second leaf (from the ear) attacked by the fungus Blumeria graminis.

The results obtained are presented in Table 2.

TABLE 2
(Test of frequency of attack)

| | % of second |
|---|---|
| Compound | leaf attacked |
| 1 | 28.0 |
| 3 | 54.0 |
| Control | 98.7 |
| A | 78.7 |
| B | 73.3 |

The results of these two tests show that the iodinated compounds of formula (I) according to the present invention are active compared with the untreated control, and also more active than the commercial reference.

### EXAMPLE OF TEST N^{o}2

This example gives an illustration of the biological efficacy of the compounds of formula (I) on pathogenic strains resistant to strobilurin derivatives.

The experiment was performed in a greenhouse.

Resistant strains were isolated and samples taken in 2001 in the north of Germany in the Bremen region in a place treated with pyraclostrobin and with picoxystrobin. These strains are characterized by their resistance to these strobilurin-type compounds. They were then kept in a controlled-environment chamber on wheat plants (Kanzler variety) treated with trifloxystrobin.

The tested compounds were sprayed on the wheat plants using solutions for application diluted in water to a volume equivalent to 250 l/ha. 24 h later, the plants were inoculated with powdery mildew (Blumeria graminis f. sp. tritici).

The extent of the disease was evaluated on each plant after 13 days of incubation.

The results obtained are assembled in Table 3 and show that the compound used allows complete control of the strains treated. These results can be applied to conditions of use in the open field.

TABLE 3

| | | Percentage of |
|---|---|---|
| | quantity | leaves affected by |
| | applied | powdery mildew |
| Compound applied | (g/ha) | (%) |
| Control plants without | — | 100 |
| treatment | | |
| Trifloxystrobin | 31 | 100 |
| (EC at 125 g/l - Twist ® | 62 | 100 |
| | 125 | 100 |
| | 250 | 100 |
| 2-ethoxy-6-iodo-3- | 31 | 0 |
| propylbenzopyran-4-one | 62 | 0 |
| (SC at 500 g/l) | | |

### EXAMPLE OF TEST N^{o}3

This example gives an illustration of the synergistic effect obtained using the compounds according to the invention in the form of a mixture with compounds of the strobilurin type, in particular trifloxystrobin, triazole, in particular fluquinconazole. The compounds according to the invention were tested in a greenhouse under preventive conditions. This synergistic effect was demonstrated on the principal diseases affecting cereals, in particular wheat and barley powdery mildews (Blumeria graminis f. sp. tritici and Blumeria graminis f. sp. hordei), wheat brown rust (Puccinia recondite) and wheat glume blotch (Septoria tritici and Septoria nodorum).

The combinations of active ingredients in Table 4 were evaluated.

TABLE 4

| Active ingredient(s) | Ratio | Applied doses (g/ha) |
|---|---|---|
| 2-ethoxy-6-iodo-3-propylbenzopyran- | — | 1.5, 3, 4, 6, 8, 12, 16, |
| 4-one (SC at 500 g/l) | | 25, 31, 50, 62 |
| fluquinconazole | — | 3, 6, 8, 12, 16, 25, 31, |
| (SC at 100 g/l - Flamenco ®) | | 50, 62, 125 |
| Trifloxystrobin | — | 4, 8, 16, 31, 62, 125 |
| (WG at 500 g/kg - Flint ®) | | |
| 2-ethoxy-6-iodo-3-propylbenzopyran- | 1/1 | |
| 4-one (SC at 500 g/l) + trifloxystrobin | 1/2.5 | |
| (WG at 500 g/kg) | 1/5 | |
| 2-ethoxy-6-iodo-3-propylbenzopyran- | 1/1 | |
| 4-one (SC at 500 g/l) + fluquinconazole | 1/2 | |
| (SC at 100 g/l) | 1/5 | |

The epoxyconazole and kresoxim-methyl mixture (SC 125 g/l+125 g/l -Ogam®) was used as reference at the recommended doses for application of 62+62 g/ha and 125+125 g/ha.

The compounds and mixtures tested were sprayed on wheat plants:

two replications,

volume for application equivalent to 250 l/ha.

On the day following the application, the wheat plants were inoculated with powdery mildew (Blumeria graminis f. sp. tritici), brown rust (Puccinia recondita), glume blotches (Septoria tritici and Septoria nodorum) and the barley plants with powdery mildew (Blumeria graminis f. sp. hordei). Once the diseases had developed, the evaluation was made by determining the extent of disease per pot.

The level of synergy was calculated using the Colby formula in order to calculate the theoretical efficacy and to compare it to the observed efficacy:

Theoretical efficacy of A+B=efficacy of A (%)+efficacy of B (%)−[efficacy of A (%)×efficacy of B (%)/100]

If the observed efficacy is higher than the theoretical efficacy, a synergistic effect is demonstrated; if it is equal, an additive effect is demonstrated; if it is lower, an antagonist effect is demonstrated.

The results obtained for a mixture of compound of formula (I) according to the invention and fluquinconazole for controlling wheat powdery mildew are assembled in Table 5.

TABLE 5

| | Dose for | | Observed | Theoretical | |
|---|---|---|---|---|---|
| | application | | efficacy | efficacy | Level |
| Active ingredient(s) | (g/ha) | Ratio | (%) | (%) | of synergy |
| 2-ethoxy-6-iodo-3- | 6 | — | 25 | — | — |
| propylbenzopyran-4-one | | | | | |
| fluquinconazole | 6 | — | 0 | — | — |
| | 12 | — | 0 | — | — |
| | 31 | — | 0 | — | — |
| 2-ethoxy-6-iodo-3- | 6 + 6 | 1/1 | 93 | 25 | +68 |
| propylbenzopyran-4-one + | 6 + 12 | 1/2 | 90 | 25 | +65 |
| fluquinconazole | 6 + 31 | 1/5 | 95 | 25 | +70 |

The results obtained for a mixture of compound of formula (I) according to the invention and fluquinconazole for controlling barley powdery mildew are assembled in Table 6.

TABLE 6

| | Dose for | | Observed | Theoretical | |
|---|---|---|---|---|---|
| | application | | efficacy | efficacy | Level |
| Active ingredient(s) | (g/ha) | Ratio | (%) | (%) | of synergy |
| 2-ethoxy-6-iodo-3- | 12 | — | 50 | — | — |
| propylbenzopyran-4-one | | | | | |
| fluquinconazole | 12 | — | 25 | — | — |
| | 25 | — | 25 | — | — |
| | 62 | — | 75 | — | — |
| 2-ethoxy-6-iodo-3- | 12 + 12 | 1/1 | 90 | 62.5 | +28 |
| propylbenzopyran-4-one + | 12 + 25 | 1/2 | 95 | 62.5 | +33 |
| fluquinconazole | 12 + 62 | 1/5 | 98 | 87.5 | +11 |

The results obtained for a mixture of compound of formula (I) according to the invention and fluquinconazole for controlling wheat brown rust are assembled in Table 7.

TABLE 7

| | Dose for | | Observed | Theoretical | |
|---|---|---|---|---|---|
| | application | | efficacy | efficacy | Level |
| Active ingredient(s) | (g/ha) | Ratio | (%) | (%) | of synergy |
| 2-ethoxy-6-iodo-3- | 50 | — | 0 | — | — |
| propylbenzopyran-4-one | | | | | |
| fluquinconazole | 100 | — | 60 | — | — |
| 2-ethoxy-6-iodo-3- | 50 + 100 | 1/2 | 90 | 60 | +30 |
| propylbenzopyran-4-one + | | | | | |
| fluquinconazole | | | | | |

The results obtained for a mixture of compound of formula (I) according to the invention and fluquinconazole for controlling wheat glume blotch (Septoria tritici) are assembled in Table 8.

TABLE 8

| | Dose for | | Observed | Theoretical | |
|---|---|---|---|---|---|
| | application | | efficacy | efficacy | Level |
| Active ingredient(s) | (g/ha) | Ratio | (%) | (%) | of synergy |
| 2-ethoxy-6-iodo-3- | 50 | — | 0 | — | — |
| propylbenzopyran-4-one | | | | | |
| fluquinconazole | 100 | — | 60 | — | — |
| 2-ethoxy-6-iodo-3- | 50 + 100 | 1/2 | 90 | 60 | +30 |
| propylbenzopyran-4-one + | | | | | |
| fluquinconazole | | | | | |

The results obtained for a mixture of compound of formula (I) according to the invention and fluquinconazole for controlling wheat glume blotch (Septora nodorum) are assembled in Table 9.

TABLE 9

| | Dose for | | Observed | Theoretical | |
|---|---|---|---|---|---|
| | application | | efficacy | efficacy | Level |
| Active ingredient(s) | (g/ha) | Ratio | (%) | (%) | of synergy |
| 2-ethoxy-6-iodo-3- | 50 | — | 0 | — | — |
| propylbenzopyran-4-one | | | | | |
| fluquinconazole | 100 | — | 75 | — | — |
| 2-ethoxy-6-iodo-3- | 50 + 100 | 1/2 | 95 | 75 | +20 |
| propylbenzopyran-4-one + | | | | | |
| fluquinconazole | | | | | |

These results demonstrate a high synergistic effect of the mixtures of compound of formula (i) and fluquinconazole in ratios of 1/1, 1/2 or 1/5 against wheat and barley powdery mildews, wheat brown rust and glume blotches. These mixtures make it possible to obtain excellent results of efficacy at agronomic doses for application.

The results obtained for a mixture of compound of formula (I) according to the invention and trifloxystrobin for controlling wheat powdery mildew are assembled in Table 10.

TABLE 10

| | Dose for | | Observed | Theoretical | |
|---|---|---|---|---|---|
| | application | | efficacy | efficacy | Level |
| Active ingredient(s) | (g/ha) | Ratio | (%) | (%) | of synergy |
| 2-ethoxy-6-iodo-3- | 6 | — | 25 | | |
| propylbenzopyran-4-one | 16 | — | 50 | — | — |
| | 25 | — | 75 | | |
| trifloxystrobin | 16 | — | 30 | — | — |
| | 31 | — | 40 | | |
| | 62 | — | 50 | | |
| 2-ethoxy-6-iodo-3- | 16 + 16 | 1/1 | 95 | 65 | +30 |
| propylbenzopyran-4-one + | 25 + 62 | 1/2.5 | 95 | 87.5 | +8 |
| trifloxystrobin | 6 + 31 | 1/5 | 85 | 55 | +30 |

The results obtained for a mixture of compound of formula (I) according to the invention and trifloxystrobin for controlling barley powdery mildew are assembled in Table 11.

TABLE 11

| | Dose for | | Observed | Theoretical | |
|---|---|---|---|---|---|
| | application | | efficacy | efficacy | Level of |
| Active ingredient(s) | (g/ha) | Ratio | (%) | (%) | synergy |
| 2-ethoxy-6-iodo-3- | 16 | — | 50 | — | — |
| propylbenzopyran-4-one | 25 | — | 75 | | |
| trifloxystrobin | 16 | — | 0 | — | — |
| | 62 | — | 0 | | |
| 2-ethoxy-6-iodo-3- | 16 + 16 | 1/1 | 75 | 50 | +25 |
| propylbenzopyran-4-one + | 25 + 62 | 1/2.5 | 85 | 75 | +10 |
| trifloxystrobin | | | | | |

These results demonstrate a high synergistic effect of the mixtures of compound of formula (I) and trifloxystrobin in ratios of 1/1, 1/2.5 or 1/5 against wheat and barley powdery mildews (on strains sensitive to compounds of the strobilurin type).

1. Compound of formula (I): 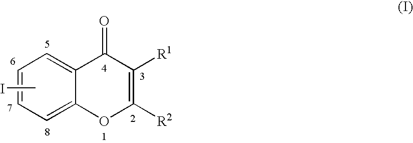
in which:
the iodine atom is placed in the 5, 6, 7 or 8 position;
R′ is chosen from a halogen atom, a substituted or unsubstituted C1-C6 10 alkyl radical, a substituted or unsubstituted C1-C6 alkenyl radical and a substituted or unsubstituted C1-C6 alkynyl radical;
R² is chosen from a substituted or unsubstituted C1-C6 alkyl radical, a substituted or unsubstituted C1-C6 alkenyl radical and a substituted or unsubstituted C1-C6 alkynyl radical, a 3- to 7-membered carbo- or heterocycle which is substituted or unsubstituted and which may be saturated, unsaturated or aromatic, a halogen atom, the cyano radical, the radical —W—R³;
W is chosen from oxygen, sulphur or the radical —NR⁴;
R³ and R⁴, which are identical or different, are chosen, independently of each other, from the hydrogen atom, a substituted or unsubstituted C1-C6 alkyl radical, a substituted or unsubstituted C1-C6 alkenyl radical and a substituted or unsubstituted C1-C6 alkynyl radical, an alkoxy radical, an amino radical, a 3- to 7-membered carbo- or heterocycle which is substituted or unsubstituted and which may be saturated, unsaturated or aromatic, R³ and R⁴ may form together a 5- to 7-membered heterocycle which may be saturated, unsaturated or aromatic, and which may also comprise another heteroatom;
and their possible geometric and/or optical isomers, in pure form or in the form of mixtures, in any proportions, including the racemic mixture(s), their possible N-oxides, addition salts with an acid, and their possible metal or metalloid complexes; R³ being different from the methyl radical or the butyl radical when the iodine is in the 6-position, when R¹ is the n-propyl radical and when W represents oxygen.

2. Compound according to claim 1, for which the iodine atom is in the 6-position.

3. Compound according to claim 1, for which R² is a radical —W—R³.

4. Compound according to claim 3, for which W represents oxygen.

5. Compound according to claim 1, for which R³ is a C1-C6 alkyl radical, a C1-C6 alkenyl radical or a C1-C6 alkynyl radical.

6. Compound according to claim 1, for which R′ is a C1-C6 alkyl radical.

7. Compound according to claim 6, for which R′ is a n-propyl radical.

8. Method for preparing a compound of formula (I) 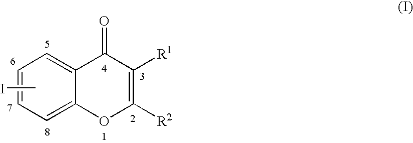
in which
the iodine atom is placed in the 5, 6, 7 or 8 position;
R′ is chosen from a halogen atom, a substituted or unsubstituted C1-C6 alkyl radical, a substituted or unsubstituted C1-C6 alkenyl radical and a substituted or unsubstituted C1-C6 alkynyl radical;
R² is chosen from a substituted or unsubstituted C1-C6 alkyl radical, a substitutedor unsubstituted C1-C6 alkenyl radical and a substituted or unsubstituted C1-C6 alkynyl radical, a 3- to 7-membered carbo- or heterocycle which is substituted or unsubstituted and which may be saturated, unsaturated or aromatic, a halogen atom, the cyano radical, the radical —W—R³; —W is chosen from oxygen, sulphur or the radical —NR⁴;
R³ and R⁴, which are identical or different, are chosen, independently of each other, from the hydrogen atom, a substituted or unsubstituted C1-C6 alkyl radical, a substituted or unsubstituted C1-C6 alkenyl radical and a substituted or unsubstituted C1-C6 alkynyl radical, an alkoxy radical, an amino radical, a 3- to 7-membered carbo- or heterocycle which is substituted or unsubstituted and which may be saturated, unsaturated or aromatic, R³ and R⁴ may form together a 5- to 7-membered heterocycle which may be saturated, unsaturated or aromatic, and which may also comprise another heteroatom;
and their possible geometric and/or optical isomers, in pure form or in the form of mixtures, in any proportions, including the racemic mixture(s), their possible N-oxides, addition salts with an acid, and their possible metal or metalloid complexes; comprising the following steps:

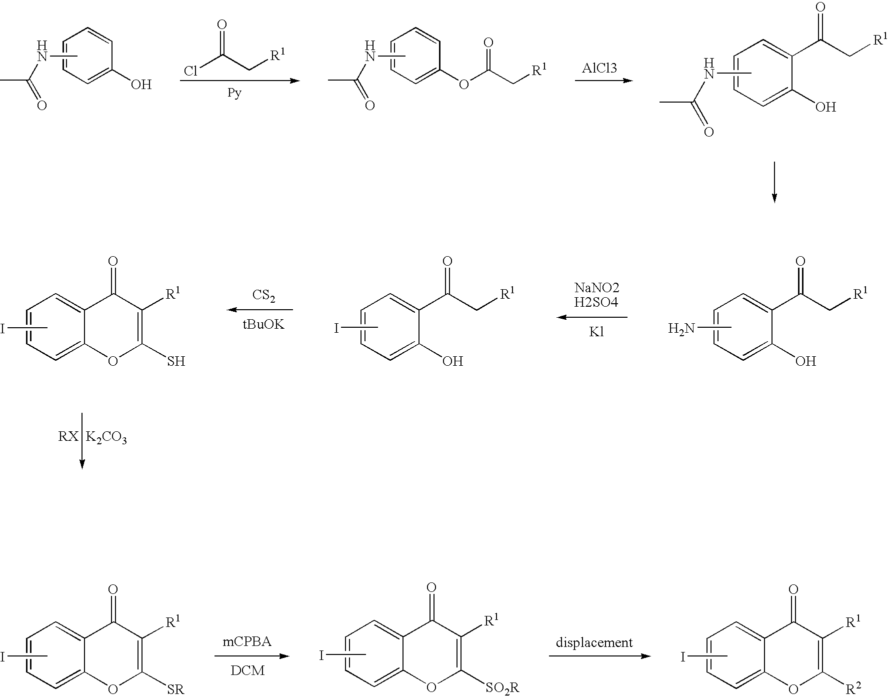

9. Fungicidal composition comprising
a) a compound of formula (I): 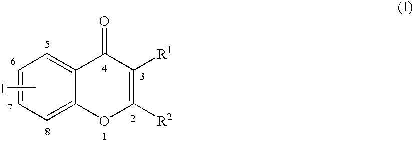
in which:
the iodine atom is placed in the 5, 6, 7 or 8 position;
R′ is chosen from a halogen atom, a substituted or unsubstituted C1-C6 alkyl radical, a substituted or unsubstituted C1-C6 alkenyl radical and a substituted or unsubstituted C1-C6 alkynyl radical;
R² is chosen from a substituted or unsubstituted C1-C6 alkyl radical, a substituted or unsubstituted C1-C6 alkenyl radical and a substituted or unsubstituted C1-C6 alkynyl radical, a 3- to 7-membered carbo- or heterocycle
which is substituted or unsubstituted and which may be saturated, unsaturated or aromatic, a halogen atom, the cyano radical, the radical —W—R³;
W is chosen from oxygen, sulphur or the radical —NR⁴;
R³ and R⁴, which are identical or different, are chosen, independently of each other, from the hydrogen atom, a substituted or unsubstituted C1-C6 alkyl radical, a substituted or unsubstituted C1-C6 alkenyl radical and a substituted or 15 unsubstituted C1-C6 alkynyl radical, an alkoxy radical, an amino radical, a 3- to 7-membered carbo- or heterocycle which is substituted or unsubstituted and which may be saturated, unsaturated or aromatic, R³ and R⁴ may form together a 5- to 7-membered heterocycle which may be saturated, unsaturated or aromatic, and which may also comprise another heteroatom;
and their possible geometric and/or optical isomers, in pure form or in the form of mixtures, in any proportions, including the racemic mixture(s), their possible N-oxides, addition salts with an acid, and their possible metal or metalloid complexes;
b) at least one other fungicidal compound.

10. Composition according to claim 9, comprising a compound of formula (I) in which the iodine atom is in the 6-position.

11. Composition according to claim 9, comprising a compound of formula (I) in which R² is a radical —W—R³.

12. Composition according to claim 11, comprising a compound of formula (I) in which W represents oxygen.

13. Composition according to claim 11, comprising a compound of formula (I) in which R³ is a radical chosen from a C1-C6 alkyl radical, a C1-C6 alkenyl radical or a C1-C6 alkynyl radical.

14. Composition according to claim 9, comprising a compound of formula (I) in which R¹ is a C1-C6 alkyl radical.

15. Composition according to claim 14, comprising a compound of formula (I) in which R¹ is a n-propyl radical.

16. Composition according to claim 9, comprising, as compound b) a fungicidal compound chosen from
b1) compounds capable of inhibiting the transport of electrons in the mitochondrial ubiquinol:ferricytochrome-c oxidoreductase respiratory chain of phytopathogenic fungal organisms, or
b2) the compounds capable of inhibiting the biosynthesis of ergosterol.

17. Composition according to claim 16, comprising as compound b1) a fingicidal compound chosen from strobilurin derivatives, fenamidone or famoxadone.

18. Composition according to claim 17, comprising a strobilurin derivative chosen from azoxystrobin, dimoxystrobin, fluoxastrobin, kresoximmethyl, pycoxystrobin, pyraclostrobin, trifloxystrobin.

19. Composition according to claim 16, comprising as compound b2) a triazole-type fungicidal compound.

20. Composition according to claim 19, comprising a triazole-type compound chosen from bromuconazole, epoxyconazole, fluquinconazole, prochloraz, prothioconazole, tebuconazole, triadimefon, triadimenol, triticonazole.

21. Composition according to claim 16, comprising as compound b) a compound chosen from cyprodinil, dinocap, fenpropidin, fenpropimorph, fosetyl, iprovalicarb, quinoxyfen, spiroxamine.

22. Method for controlling phytopathogenic fungi of crops at a site which is infested or which may be infested by them, which comprises the application to the said site of at least one compound of formula (I) according to claim 1.

23. Method for controlling phytopathogenic fungi of crops at a site which is infested or which may be infested by them, which comprises the application to the said site of at least one composition according to claim 9.

24. Compound according to claim 2, for which R² is a radical —W—R³.

25. Composition according to claim 10, comprising a compound of formula (I) in which R² is a radical —W—R³.

26. Composition according to claim 12, comprising a compound of formula (I) in which R³ is a radical chosen from a C1-C6 alkyl radical, a C1-C6 alkenyl radical or a C1-C6 alkynyl radical.
